(19) United States
(12) Patent Application Publication
(10) Pub. No.: US 2024/0047021 A1
(43) Pub. Date: Feb. 8, 2024

(54) MEDICAL INFORMATION PROCESSING APPARATUS, MEDICAL INFORMATION PROCESSING METHOD, AND STORAGE MEDIUM

(71) Applicant: CANON MEDICAL SYSTEMS CORPORATION, Otawara-shi (JP)

(72) Inventors: Yusuke KANO, Nasushiobara (JP); Anri YAMAZAKI, Nasushiobara (JP); Yohei MURAGUCHI, Nasushiobara (JP)

(73) Assignee: CANON MEDICAL SYSTEMS CORPORATION, Otawara-shi (JP)

(21) Appl. No.: 18/364,563

(22) Filed: Aug. 3, 2023

(30) Foreign Application Priority Data
Aug. 8, 2022 (JP) 2022-126297

(51) Int. Cl. G16H 10/20; G16H 50/70

(52) U.S. Cl. CPC G16H 10/20; G16H 50/70; G16H 10/60

(57) ABSTRACT

A medical information processing apparatus of an embodiment includes processing circuitry. The processing circuitry acquires symptom reply information regarding a predetermined symptom of a patient. The processing circuitry acquires condition data of the patient related to the symptom. The processing circuitry identifies a condition sensitivity component of the symptom reply information on the basis of change in the symptom reply information with respect to change in the condition data.

### CROSS-REFERENCE TO RELATED APPLICATION

The present application claims priority based on Japanese Patent Application No. 2022-126297 filed Aug. 8, 2022, the content of which is incorporated herein by reference.

### BACKGROUND OF THE INVENTION

### Field of the Invention

Embodiments of the present invention relate to a medical information processing apparatus, a medical information processing method, and a storage medium.

### Description of Related Art

In clinical situations in which types of data that can be acquired are limited, such as remote monitoring of heart failure patients, subjective symptoms that patients reply with on a daily basis are important information for medical staff such as doctors and clinical decision support (CDS) systems to ascertain disease states more accurately. In this regard, conventionally, in order to correct differences in interpretation of subjects in results of a questionnaire about a product, there is a known method of separating all components of interpretations with respect to a target product into individual difference components of interpretation of each subject. However, in the case of a questionnaire about subjective symptoms of a patient, tendencies of replies change according to a patient's own disease state which changes from moment to moment, and thus it is impossible to separate component information of replies through the conventional method. Accordingly, conventionally, there are cases where it is impossible to appropriately identify the presence or absence of various factors and the factors in symptoms of a patient affected by the factors.

### BRIEF DESCRIPTION OF THE DRAWINGS

FIG. 1 is a diagram showing an example of a configuration of a medical information processing system including a medical information processing apparatus according to an embodiment.

FIG. 2 is a diagram showing an example of processing in an identification function.

FIG. 3 is a diagram showing an example of processing of the identification function at the time of identifying a biased tendency component.

FIG. 4 is a diagram showing an example of processing of the identification function at the time of identifying a random tendency component.

FIG. 5 is a diagram showing an example of a first image generated by an image generation function.

FIG. 6 is a diagram showing an example of a second image generated by the image generation function.

FIG. 7 is a diagram showing an example of a third image generated by the image generation function.

FIG. 8 is a flowchart showing a series of processing executed by processing circuitry.

FIG. 9 is a diagram showing an example of a configuration of processing circuitry in a modified example.

FIG. 10 is a diagram showing an example of processing of a question generation function.

FIG. 11 is a diagram showing generation of a plurality of questions in different questioning manners.

FIG. 12 is a diagram showing an example of an image displayed on a terminal device.

FIG. 13 is a diagram showing an example of an image including content of a question in a different questioning manner from FIG. 12.

FIG. 14 is a diagram showing generation of content of a question for adjusting component information.

### DETAILED DESCRIPTION OF THE INVENTION

A medical information processing apparatus, a medical information processing method, and a storage medium according to embodiments will be described below with reference to the drawings.

A medical information processing apparatus of an embodiment includes processing circuitry. The processing circuitry acquires symptom reply information regarding a predetermined symptom of a patient. The processing circuitry acquires patient condition data related to the symptom. The processing circuitry identifies a condition sensitivity component of the symptom reply information on the basis of change in the symptom reply information with respect to changes in the condition data.

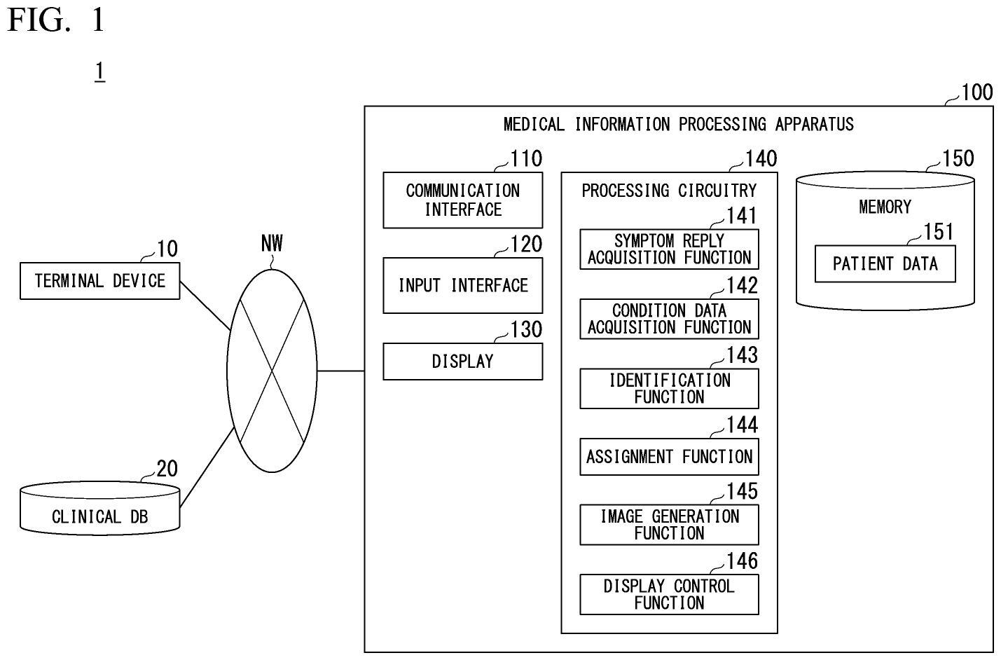

FIG. 1 is a diagram showing an example of a configuration of a medical information processing system 1 including a medical information processing apparatus according to an embodiment. The medical information processing system 1 includes, for example, a terminal device 10, a clinical DB 20, and a medical information processing apparatus 100. The terminal device 10, the clinical DB 20, and the medical information processing apparatus 100 are connected via a network NW, for example, such that they can communicate. At least one of the terminal device 10 and the clinical DB 20 may be provided in plural in the medical information processing system 1.

The network NW indicates general information communication networks using telecommunication technology. The network NW includes a wireless/wired local area network (LAN), a wide area network (WAN), an Internet network, a telephone communication network, an optical fiber communication network, a cable communication network, a satellite communication network, and the like.

The terminal device 10 receives input of symptom reply information regarding a predetermined symptom from a patient (an example of a subject), for example. Predetermined symptoms include, for example, not only symptoms regarding diseases (sicknesses) but also symptoms regarding biometric data of a patient, such as a blood pressure, a body temperature, a pulse, and blood data. For example, the terminal device receives an input (reply result) from the patient with respect to a subjective symptom questionnaire regarding the predetermined symptom, acquired from the medical information processing apparatus 100 or another external device. Further, the terminal device 10 may be a terminal device 10 used by a medical staff member (for example, a doctor or a nurse) who treats the patient. In this case, the medical staff member orally inquires about subjective symptoms to the patient, and the terminal device 10 receives results of the inquiry about subjective symptoms input by the medical staff member. In addition, the terminal device 10 may acquire patient condition data (for example, a blood pressure value, a body temperature, a pulse rate, the numbers of cells, enzymes, antibodies, and the like contained in the blood) regarding the predetermined symptom.

The terminal device 10 transmits the acquired data to the medical information processing apparatus 100 via the network NW. Further, the terminal device 10 may transmit the acquired data to the clinical DB 20. The terminal device 10 may be, for example, a terminal device included in a CDS system.

The terminal device 10 is, for example, a smartphone, a tablet terminal, a general-purpose personal computer (PC), or a server device.

The clinical DB 20 is a database in which symptom reply information regarding predetermined symptoms, condition data related to predetermined symptoms of patients, and the like are stored. The data stored in the medical care DB 20 may include not only data acquired from the terminal device 10 but also information acquired from other medical devices capable of acquiring patient condition data and the like. Further, the clinical DB 20 may store data obtained from the medical information processing apparatus 100 via the network NW. The clinical DB 20 may be, for example, a general-purpose DB server or a cloud server.

The medical information processing apparatus 100 analyzes and evaluates the condition of a patient, for example, on the basis of reply information (symptom reply information) acquired multiple times at different timings with respect to a predetermined symptom of the patient. The medical information processing apparatus 100 displays processing results on its own display, transmits the processing results to the terminal device 10 via the network NW or transmits them to the clinical DB 20. The medical information processing apparatus 100 may be, for example, a general-purpose PC, a server device, or a cloud server.

Here, a functional configuration of the medical information processing apparatus 100 will be described. The medical information processing apparatus 100 includes, for example, a communication interface 110, an input interface 120, a display 130, processing circuitry 140, and a memory 150.

The communication interface 110 includes, for example, a communication interface such as a network interface controller (NIC). The communication interface 110 communicates with external devices such as the terminal device 10 and the clinical DB 20 via the network NW and outputs acquired information to the processing circuitry 140 and the like. In addition, the communication interface 110 transmits information to external devices such as the terminal device 10 and the clinical DB 20 connected via the network NW under the control of the processing circuitry 140.

The input interface 120 receives various input operations from a user, converts the received input operations into electrical signals, and transmits the electrical signals to the processing circuitry 140. For example, when an input operation is performed by the user, the input interface 120 generates information according to the input operation. The input interface 120 transmits the generated information according to the input operation to the processing circuitry 140. The input interface 120 is realized by, for example, a mouse, a keyboard, a trackball, a switch, buttons, a joystick, a touch panel, and the like. Further, the input interface 120 may be realized by, for example, a user interface that receives voice input such as a microphone. When the input interface 120 is a touch panel, the display 130 which will be described later may be formed integrally with the input interface 120.

The display 130 displays various types of information. For example, the display 130 displays an image generated by the processing circuitry 140, a graphical user interface (GUI) for receiving various input operations from the user, and the like. For example, the display 130 is a liquid crystal display (LCD), a cathode ray tube (CRT) display, an organic electroluminescence (EL) display, or the like.

The processing circuitry 140 includes, for example, a symptom reply acquisition function 141, a condition data acquisition function 142, an identification function 143, an assignment function 144, an image generation function 145, and a display control function 146. The processing circuitry 140 realizes these functions by, for example, a hardware processor executing a program stored in a storage device (storage circuit).

The hardware processor means, for example, circuitry such as a central processing unit (CPU), a graphics processing unit (GPU), an application specific integrated circuit (ASIC), a programmable logic device (for example, a simple programmable logic device (SPLD), a complex programmable logic device (CPLD), or a field programmable gate array (FPGA)), or the like.

Instead of storing the program in the storage device, the program may be configured to be directly embedded in the circuit of the hardware processor. In this case, the hardware processor realizes the function thereof by reading and executing the program embedded in the circuit. The aforementioned program may be stored in a storage device in advance, or may be stored in a non-transitory storage medium such as a DVD or CD-ROM and installed into the storage device from the non-transitory storage medium when the non-transitory storage medium is set in a drive device (not shown)) of the medical information processing apparatus 100.

The hardware processor is not limited to being configured as a single circuit, and may be configured as a single hardware processor by combining multiple independent circuits to realize each function. Further, a plurality of components may be integrated into one hardware processor to realize each function.

The memory 150 is realized by, for example, a semiconductor memory such as a random access memory (RAM) and a flash memory, a hard disk, an optical disc, or the like. These non-transitory storage media may be realized by other storage devices such as a network attached storage (NAS) and external storage server devices connected via the network NW. Moreover, these non-transitory storage media may be realized by storage devices such as a read only memory (ROM) and a register. The memory 150 stores, for example, patient data 151, programs, and other various types of information. The patient data 151 includes symptom reply information regarding predetermined symptoms of patients and patient condition data related to the predetermined symptoms acquired from the terminal device 10 or the clinical DB 20. Such information is managed chronologically for each patient.

The symptom reply acquisition function 141 acquires symptom reply information regarding a predetermined symptom of a patient transmitted from the terminal device 10 or the clinical DB 20 via the communication interface 110. Further, the symptom reply acquisition function 141 may acquire symptom reply information through input from the input interface 120.

The condition data acquisition function 142 acquires patient condition data related to a predetermined symptom transmitted from the terminal device 10 or the clinical DB 20 via the communication interface 110. The condition data acquisition function 142 may acquire patient condition data through input from input interface 120. The symptom reply acquisition function 141 and the condition data acquisition function 142 may acquire information from one or both of the terminal device 10 and the clinical DB 20. Information acquired by the symptom reply acquisition function 141 and the condition data acquisition function 142 may be stored in the memory 150 as the patient data 151.

The identification function 143 identifies component information of symptom reply information acquired by the symptom reply acquisition function 141 on the basis of change in the symptom reply information with respect to change in condition data acquired by the condition data acquisition function 142. Component information is, for example, a condition sensitivity component. A condition sensitivity component is, for example, an index value indicating the magnitude of change in symptom reply information with respect to change in condition data, and the greater the change in the symptom reply information with respect to the change in the condition data, the higher the value of the condition sensitivity component.

For example, the identification function 143 identifies a condition sensitivity component of a symptom reply on the basis of change in the symptom reply with respect to change in a disease state included in condition data. For a disease state, for example, results of determination for a patient by others, measurement values of examinations, determination results in a CDS system, and the like are used. A symptom reply may be, for example, a two-level (presence and absence) value, a multi-level (e.g., Likert scale and the like) value, or a values extracted from a continuous range using a slide bar, or the like.

For example, the identification function 143 identifies a condition sensitivity component using regression, classification, analysis of variance, or the like using a disease state and symptom reply information as inputs. In the following, as an example, a condition sensitivity component is identified using an ordinal logistic regression analysis based on continuous-valued disease states (e.g., blood pressure and the like) and three-stage discrete-valued symptom replies (e.g., patient's subjective symptoms).

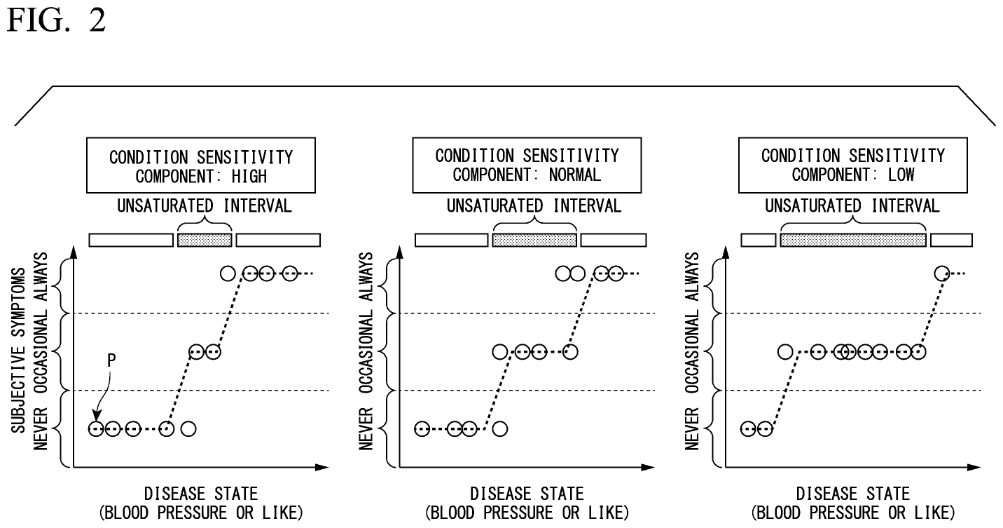

FIG. 2 is a diagram showing an example of processing in the identification function 143. In the example of FIG. 2, the horizontal axis represents the magnitude of a disease state (blood pressure and the like) and the vertical axis represents three-level categories (discrete values) of subjective symptoms of a patient: “always,” “occasional,” and “never.” A category is, for example, an option when a patient inputs a symptom reply. The categories are not limited to the example of FIG. 2. Further, the example of FIG. 2 also shows a relationship between a disease state and symptom reply information in each of cases in which a condition sensitivity component is high, normal, and low. “Normal” means a state (appropriate state) in which change in subjective symptoms with respect to change in disease information is neither high nor low and thus determined to be appropriate. In addition, each plotted point P in FIG. 2 indicates a reply result of subjective symptoms of a patient with respect to a disease state value (for example, a blood pressure value).

For example, the identification function 143 derives the probability of each category of symptom reply information from results of ordinal logistic regression and identifies a category with a maximum probability for the disease state. The identification function 143 identifies, for example, an interval of the disease state in which the symptom reply information shown in FIG. 2 is not saturated (i.e., an interval corresponding to subjective symptoms of “occasional,” referred to as a “unsaturated interval” hereinafter). Then, the identification function 143 determines whether the condition sensitivity component is high, normal, or low on the basis of the ratio of the unsaturated interval to the entire interval for which the symptom reply is obtained (hereinafter referred to as an “unsaturated interval ratio”). The identification function 143 may quantify the condition sensitivity component on the basis of, for example, the unsaturated interval ratio.

Further, at the time of identifying the condition sensitivity component, the identification function 143 may quantify the magnitude of the condition sensitivity component of the patient as a relative amount to the magnitude of the component of the entire group to which the patient belongs. A group is, for example, a group determined in advance by age, sex, period, or the like. In this case, the identification function 143 calculates an unsaturated interval ratio for the entire group to which the patient belongs, for example, regards replies of the patient for which the unsaturated interval ratio belongs to the top 10% as having a low condition sensitivity component, regards replies of the patient for which the unsaturated interval ratio belongs to the bottom 10% as having a high condition sensitivity component, and identities other replies of the patient as having a normal condition sensitivity component.

The method of identifying a condition sensitivity component is not limited to the above example, and the identification function 143 may identify a condition sensitivity component using, for example, the magnitude of an inclination obtained through linear regression. Further, at the time of identifying an unsaturated interval and an unsaturated interval ratio, the identification function 143 may identify the intervals on the basis of not only the intervals but also distributions of data obtained within the intervals.

Further, the identification function 143 may identify a biased tendency component that is consistent in variations in replies instead of (or in addition to) the condition sensitivity component. A biased tendency component is an example of component information indicating a biased tendency of replies of a patient. The biased tendency component is, for example, an index value indicating that a certain patient tends to reply biasedly that subjective symptoms are “always” even if a disease state changes. For example, the identification function 143 calculates a biased tendency component through regression, classification, analysis of variance, or the like using a disease state and symptom reply information as inputs. In addition, the identification function 143 may identify the based tendency component using ordinal logistic regression analysis on the basis of a continuous-valued disease state and symptom reply information regarding a three-level category (discrete value), similar to the condition sensitivity component.

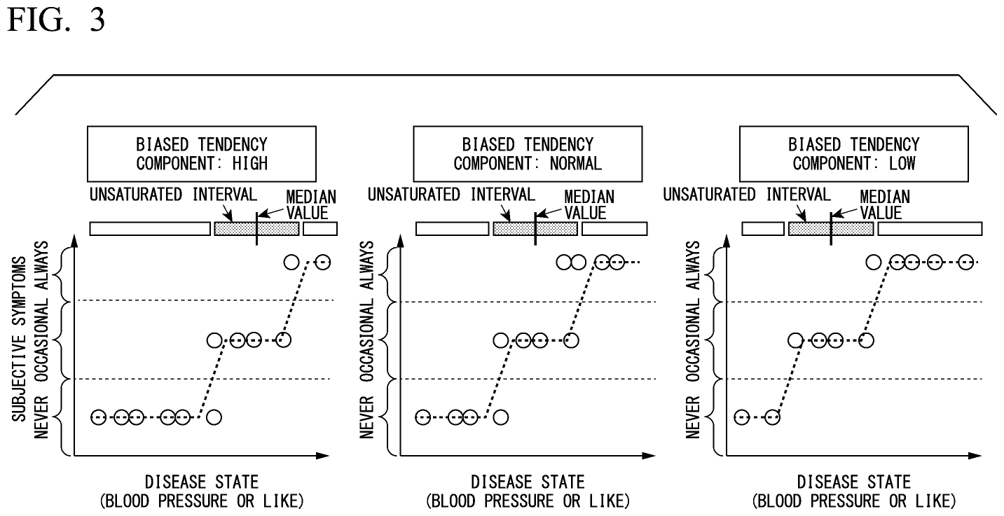

FIG. 3 is a diagram showing an example of processing of the identification function 143 at the time of identifying a biased tendency component. In the example of FIG. 3, the horizontal axis represents the magnitude of a disease state (blood pressure or the like) and the vertical axis represents three-level categories (discrete values) of subjective symptoms of a patient: “always,” “occasional,” and “never,” as in FIG. 2. The example of FIG. 3 also shows a relationship between the disease state and symptom reply information in each of cases where the biased tendency component is high, normal, and low. For example, the identification function 143 determines the biased tendency component on the basis of the median value of an unsaturated interval with respect to the entire interval for which the symptom reply information is obtained.

For example, the identification function 143 identifies the biased tendency component as high if the median value of a saturation interval is greater than the center of a predetermined blood pressure range (including blood pressure ranges before and after the center), identifies the biased tendency component as normal if the median value is included in the center, and identifies the biased tendency component as low if the median value is less than the center. Further, the identification function 143 may quantify the magnitude of component information of a patient as a relative amount to the magnitude of the component of the entire group to which the patient belongs, similar to the condition sensitivity component.

Further, the identification function 143 may identify a random tendency component instead of (or in addition to) the condition sensitivity component and the biased tendency component. The random trend component is an example of component information indicating a degree of randomness that is not consistent in variations in replies. The random tendency component is, for example, an index value indicating how much subjective symptoms of a patient vary (in other words, whether or not symptoms are correlated) depending on a predetermined disease state.

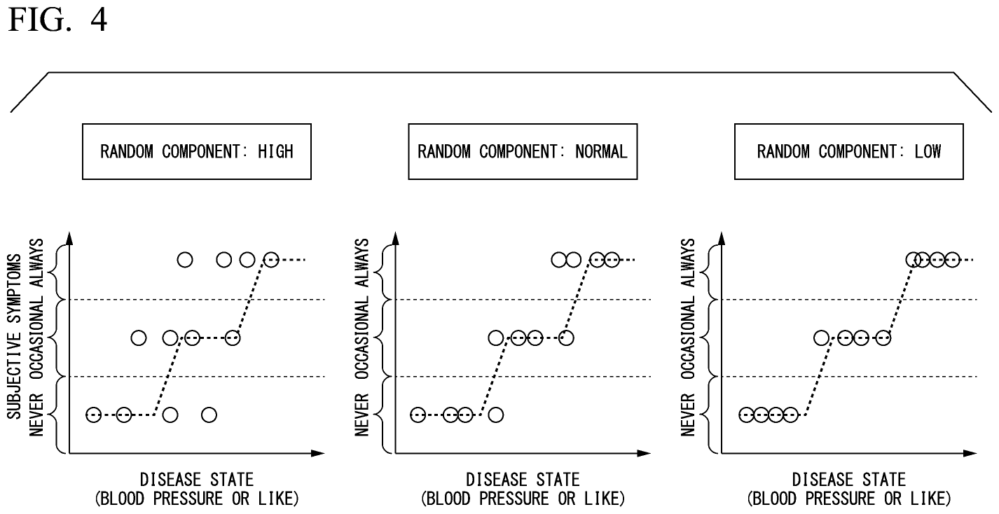

FIG. 4 is a diagram showing an example of processing of the identification function 143 at the time of identifying a random tendency component. In the example of FIG. 4, the horizontal axis represents the magnitude of a disease state (blood pressure or the like) and the vertical axis represents three-level categories (discrete values) of subjective symptoms of a patient: “always,” “occasional,” and “never,” as in FIG. 2. The example of FIG. 4 also shows a relationship between the disease state and symptom reply information in each of cases where the random component is high, normal, and low.

For example, the identification function 143 determines the random tendency component on the basis of a degree of fittingness of ordinal logistic regression serving as a predetermined criterion (e.g., standard error of an estimator). For example, the identification function 143 sets a reference transition (dotted line in the drawing) of subjective symptoms with respect to the magnitude of a blood pressure or the like and identifies the random tendency component depending on how much actual subjective symptoms of the patient have varied with respect to the set transition (whether the actual subjective symptoms have deviated from the reference transition). For example, the identification function 143 identifies the random component as high if the degree of variation is greater than a predetermined range, identifies the random component as normal if the degree of variation is within the predetermined range, and identifies the random component as low if the degree of variation is less than the predetermined range. The identification function 143 may identify the random component by the number of subjective symptom plots P of the patient that have deviated from the reference transition. In addition, the identification function 143 may quantify the magnitude of the component of the patient as a relative amount to the magnitude of the component of the entire group to which the patient belongs, similar to the condition sensitivity component and the biased tendency component.

The assignment function 144 assigns information on component information identified by the identification function 143 to symptom reply information. For example, the assignment function 144 assigns an index value of a condition sensitivity component identified by the identification function 143 to each piece of symptom reply information. Further, the assignment function 144 may assign at least one of a biased tendency component and a random tendency component to symptom reply information instead of (or in addition to) the condition sensitivity component. In addition, the assignment function 144 may assign component information to individual symptom replay, symptom reply information for a specific period or all periods regarding a specific symptom, or all symptom reply information of the patient. The assignment function 144 may store the assigned information in the memory 150 or may transmit the assigned information to the terminal device 10 or the clinical DB 20 via the network NW.

The image generation function 145 generates an image including symptom reply information and component information. For example, the image generation function 145 generates an image in which at least one component information among a condition sensitivity component, a biased component, and a random component has been assigned to symptom reply information by the assignment function 144. In addition, the image generation function 145 may generate an image including a relationship between a disease state and a subjective symptom corresponding to a patient for each component information, as shown in FIGS. 2 to 4 described above.

The display control function 146 causes an image generated by the image generation function 145 to be displayed on the display 130 or to be transmitted to the terminal device 10 via the network NW. In addition, the display control function 146 may cause the image generation function 145 to generate an image, perform change of display content, or the like according to an instruction of the user. Further, the display control function 146 may store processing results and the like in the memory 150 or cause information stored in the memory 150 to be displayed on the display 130 or to be transmitted to the terminal device 10 or the clinical DB 20.

Some examples of images generated by the image generation function 145 will be described below. In addition, examples of assigning a condition sensitivity component, a biased component, and a random component to symptom reply information and displaying the symptom reply information will be described below. Further, it is assumed that images shown below are images displayed for each patient.

### First Image

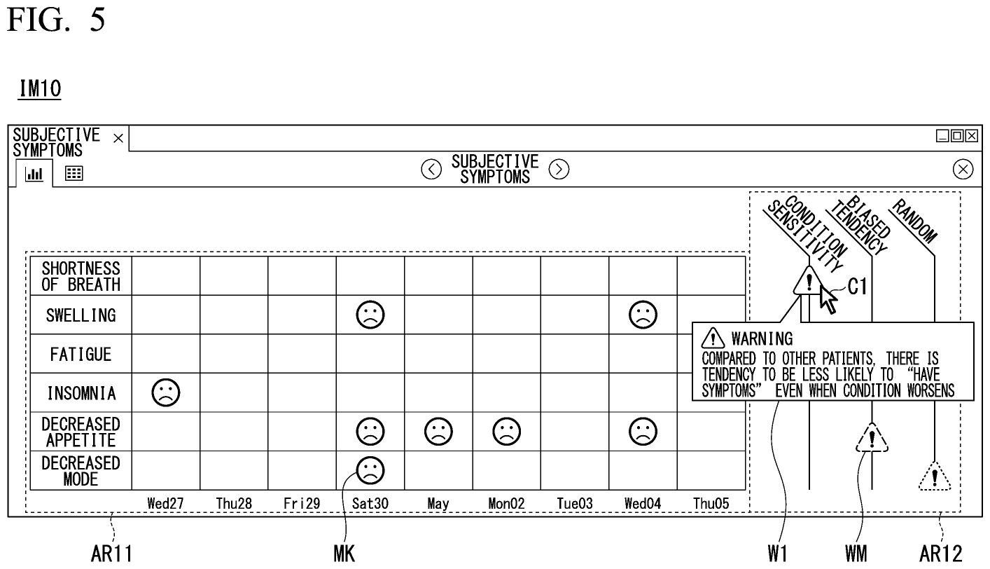

FIG. 5 is a diagram showing an example of a first image IM10 generated by the image generation function 145. The content and display modes such as a layout, color, font, and design displayed in the first image IM10, which will be described below, are not limited thereto. The same applies to other images which will be described later. The first image IM10 includes, for example, a subjective reply display area AR11 and a component information display area AR12.

In the subjective reply display area AR11, the horizontal axis represents a date (which may include days of the week) and the vertical axis represents degrees of subjective symptoms of a patient with respect to predetermined condition data (for example, shortness of breath, swelling, fatigue, insomnia, decreased appetite, decreased mood, etc.). In the first image IM10, for example, a predetermined mark MK is displayed at a portion where the patient replies that subjective symptoms are “always” for each item of predetermined symptoms. The image generation function 145 may change the type of the mark MK according to the subjective symptom category (“always”, “occasional” or “never”).

In addition, in the component information display area AR12, a warning mark WM is displayed at a position corresponding to a numerical value (index value) of each of a condition sensitivity component, a bias tendency component, and a random component. In the example of FIG. 5, the higher the numerical value, the higher the warning mark WM displayed in the figure. For example, if the numerical value is greater than a threshold value or if the user selects the warning mark WM with a cursor C1, the display control function 146 displays warning information W1 corresponding to that component. In the example of FIG. 5, as warning information for the condition sensitivity component, text information such as “There is a tendency to be less likely to “have symptoms” than other patients even when the condition worsens” is displayed. The image generation function 145 may generate and output a sound corresponding to warning information instead of (or in addition to) generating the image.

By displaying the first image IM10, it is possible to make it easier for the patient to ascertain the transition of subjective symptoms for each piece of condition data and to provide component information identified for each patient. Therefore, users such as medical staff and the like can more appropriately ascertain the presence or absence of influences of various factors and the factors in subjective symptoms of patients influenced by the factors.

### Second Image

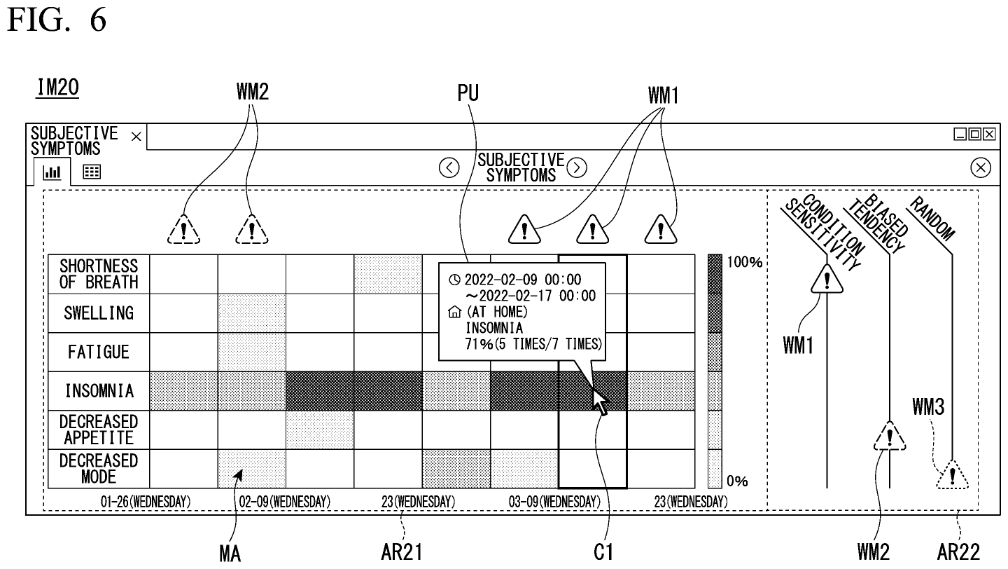

FIG. 6 is a diagram showing an example of a second image IM20 generated by the image generation function 145. The second image IM20 includes, for example, a subjective reply display area AR21 and a component information display area AR22. In the case of the second image IM20, a display method in the subjective reply display area AR21 differs from that in the subjective reply display area AR11 as compared to the first image IM10. Therefore, the following description will focus on the subjective reply display area AR21.

In the subjective reply display area AR21, the horizontal axis represents a date (which may include days of the week) and the vertical axis represents subjective symptoms when symptoms are predetermined. In addition, in the subjective reply display area AR21, areas MA corresponding to condition data for each predetermined period such as one week are defined in a matrix form, and patterns and colors corresponding to the subjective symptoms are displayed such that they can be identified for each area MA.

For example, if the user selects one of the matrix-shaped areas MA displayed in the subjective reply display area AR21 with a cursor C1, the display control function 146 displays a subjective symptom during that period as a pop-up image PU. In the example of FIG. 6, information representing that “there is a subjective symptom of insomnia 5 out of 7 times during the period from 2022/2/9 00:00:00 to 2022/02/17 00:00:00”is displayed.

Furthermore, in the subjective reply display area AR21, if there is warning information for the condition sensitivity component, biased tendency component, and random component in the periods of the horizontal axis, marks corresponding to warning marks WM1 to WM3 for the respective components displayed in the component information display area AR22 are displayed in association with periods of the subjective reply display area AR21. In the example of FIG. 6, the warning mark WM1 for the condition sensitivity component and the warning mark WM2 for the biased tendency component are displayed above the positions corresponding to the periods in the subjective reply display area AR21. When any warning mark has been selected with the cursor C1, the display control function 146 may display warning information corresponding to the warning mark.

In this way, according to the second image IM20, it is possible to ascertain the tendency of subjective symptoms of the patient over a longer period of time than the first image IM10 and how the tendency changes. Further, according to the second image IM20, it is possible to more accurately ascertain a period in which a warning regarding a subjective symptom is issued by displaying the warning mark WM.

### Third Image

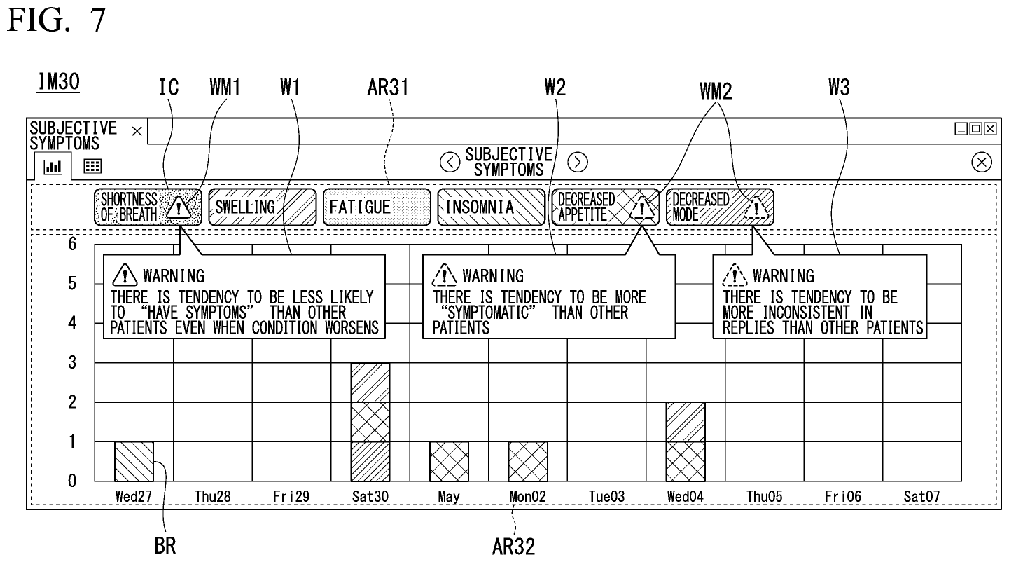

FIG. 7 is a diagram showing an example of a third image IM30 generated by the image generation function 145. In the Image IM30, for example, a symptom data list display area AR31 and a component added value display area AR32 are displayed. Icons IC corresponding to predetermined symptom data are displayed in the symptom data list display area AR31. Each icon is displayed such that it can be identified, for example, by a color or a pattern. Further, if there is warning data for symptom data in the symptom data list display area AR31, it is possible to notify the user of a tendency by displaying warning marks WM1 to WM3 or the like. In the example of FIG. 7, the warning mark WM1 for the condition sensitivity component is displayed on the icon IC of the condition data of “shortness of breath” and the warning mark WM2 for the biased tendency component is displayed on the icons IC of condition data of “decreased appetite” and “decreased mood.” The display control function 146 may display warning information W1 to W3 for the above-described warning marks.

The horizontal axis of the component added value display area AR32 represents a date (which may include days of the week) and the vertical axis represents a count number. In the third image IM30, with respect to the items displayed in the symptom item list display area AR31, a block BR of an item with a symptom is displayed as a stacked graph in the same display mode as the color and pattern displayed in the symptom item list display area AR31. In this way, according to the third image IM30, it is possible to easily ascertain when and what subjective symptoms occurred.

The image generation function 145 is not limited to generating the above-described described images IM10 to IM30 and may generate an image by combining each of the above-described images IM10 to IM30 with part or all of another image. Further, the display control function 146 may appropriately switch and display the above-described images IM10 to IM30 according to user's selection.

### Processing Flow

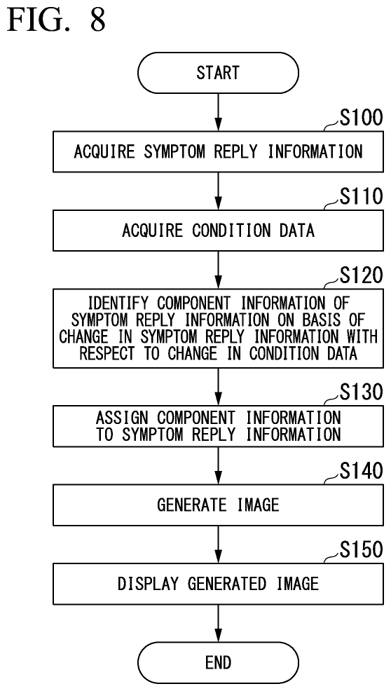

A processing flow of the processing circuitry 140 in an embodiment will be described below. FIG. 8 is a flowchart showing a series of processing executed by the processing circuitry 140. In the example of FIG. 8, the symptom reply acquisition function 141 acquires symptom reply information from the terminal device 10, the clinical DB 20, or the like via the network NW (step S100). Next, the condition data acquisition function 142 acquires condition data of a patient from the terminal device 10, the clinical DB 20, or the like (step S110).

Next, the identification function 143 identifies component information of the symptom reply information on the basis of change in the symptom reply information with respect to change in the condition data (step S120). The component information is, for example, at least one of a condition sensitivity component, a biased tendency component, and a random component. Next, the assignment function 144 assigns the component information to the symptom reply information (step S130). Next, the image generation function 145 generates an image in which the component information has been assigned to the symptom reply information (step S140). The display control function 146 causes the display 130 to display the image generated by the image generation function 145 (step S150). In the processing of step S150, the generated image may be transmitted to an external device (e.g., the terminal device 10, the clinical DB 20, or the like) via a network. Accordingly, processing of this flowchart ends.

### MODIFIED EXAMPLE

For example, in a case where a disease state of a patient satisfies specific conditions, the medical information processing apparatus 100 of the embodiment may generate a question for acquiring symptom reply information from the patient and notify the terminal device 10 or the like of the question to acquire the symptom reply information to be acquired. Hereinafter, the above-described content will be described as a modified example. The medical information processing apparatus 100 in the modified example is different in that it includes processing circuitry 140A, which will be described later, instead of the processing circuitry 140. Therefore, the following description will focus on the configuration of the processing circuitry 140A, and description of other configurations will be omitted.

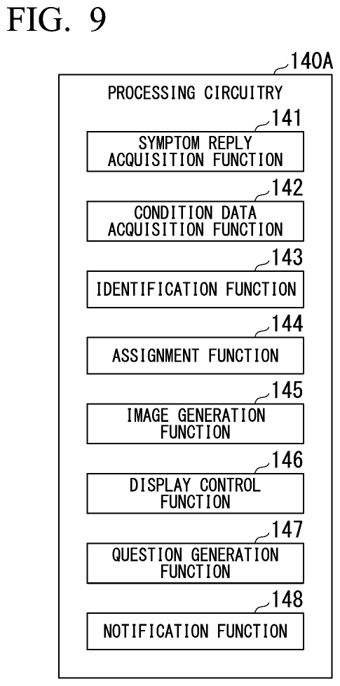

FIG. 9 is a diagram showing an example of the configuration of the processing circuitry 140A in the modified example. The processing circuitry 140A includes, for example, a symptom reply acquisition function 141, a condition data acquisition function 142, an identification function 143, an assignment function 144, an image generation function 145, a display control function 146, a question generation function 147, and a notification a function 148. The processing circuitry 140A realizes these functions by, for example, a hardware processor executing a program stored in a storage device (storage circuit). Here, the processing circuitry 140A differs from the processing circuitry 140 in that it includes the question generation function 147 and the notification function 148. Therefore, the following description will focus on the question generation function 147 and the notification function 148.

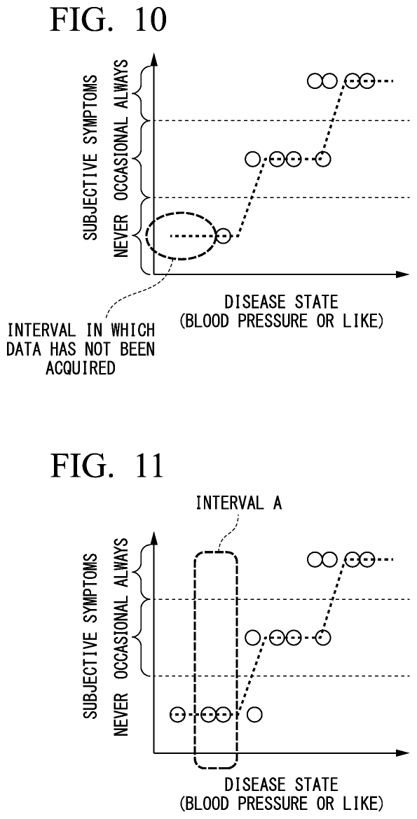

In a case where there is a symptom for which symptom reply information of a patient is insufficient as a specific condition, for example, the question generation function 147 generates a question to the patient for obtaining symptom reply information regarding the symptom. For example, in a case where there is no or symptom reply information when a disease state of the patient is a predetermined state (range), or little symptom reply information (the number of replies is equal to or less than a threshold value), the question generation function 147 generates a question to the patient for obtaining a symptom reply with respect to that part and notifies the patient of the generated question via notification function 148. FIG. 10 is a diagram showing an example of processing of the question generation function 147. In the example of FIG. a relationship of three categories (“always,” “occasional,” and “never”) with respect to a disease state (blood pressure or the like) is shown. For example, it is assumed that there is an interval in which data has not been acquired in a case where symptom reply information shown in FIG. 10 has been input. In this case, the question generation function 147 generates a question for obtaining replies to symptoms in a disease state when a patient has the disease state in that interval. In the example of FIG. 10, when the blood pressure is low (below a threshold value), a question for obtaining symptom replies of the patient is generated. Accordingly, it is possible to acquire information for more appropriately analyzing the tendency in replies of the patient.

The question generation function 147 may not generate a question for acquiring symptom reply information in a case where sufficient symptom reply information regarding the disease state of the patient has been acquired (a predetermined number or more has been acquired). Accordingly, it is possible to reduce an extra burden of causing the patient to input replies.

In addition, the question generation function 147 may generate a plurality of questions in different questioning manners for similar disease states within a predetermined interval. FIG. 11 is a diagram showing generation of a plurality of questions in different questioning manners. For example, as shown in FIG. 11, in a case where a disease state is within a predetermined interval A, the question generation function 147 generates a question with substantially the same content but in a different way. The predetermined interval A is, for example, an interval in which subjective symptoms of a patient are at the same level (category), and in the example of FIG. 11, the subjective symptoms are in the range of “never,” but the range is not limited thereto. For example, if a plurality of questions with different questioning manners are generated in an interval of a disease state in which the subjective symptoms are “occasional,” the patient's impression of the questions will also change, and thus it is possible to obtain different replies such as “always” and “never.” As a result, an unsaturated interval of the patient for the subjective symptoms can be reduced.

The notification function 148 transmits question information generated by the question generation function 147 to the terminal device 10 used by the patient. In this case, the image generation function 145 may generate an image including the question information and information for causing replies to the question to be input and may cause the notification function 148 to transmit the generated image to the terminal device 10.

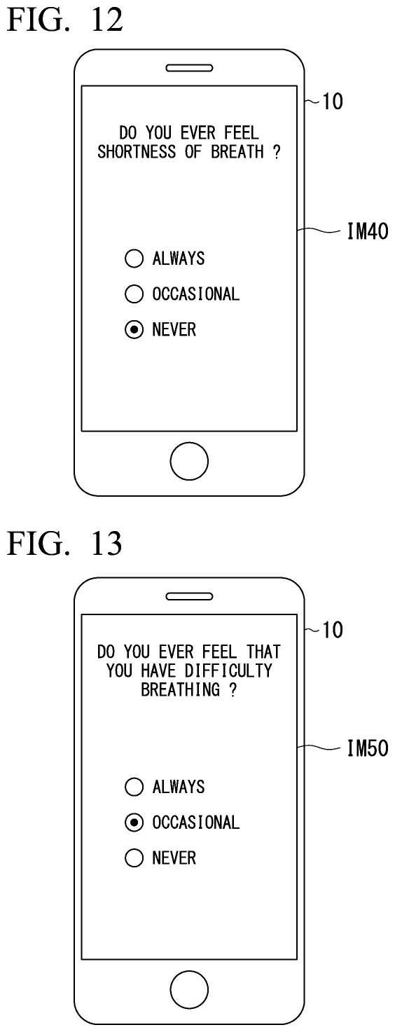

FIG. 12 is a diagram showing an example of an image IM40 displayed on the terminal device 10. The image IM40 is an image generated by the question generation function 147. The image IM40 displays text representing the content of a question such as “Do you ever feel shortness of breath?” with respect to a symptom item of “shortness of breath” and a selection area including radio buttons for selecting one of categories (options) of “always,” “occasional,” and “never”. The image IM40 may include a send button or the like for sending a selected result. By displaying such an image, it is possible to easily ascertain a reply to the generated question.

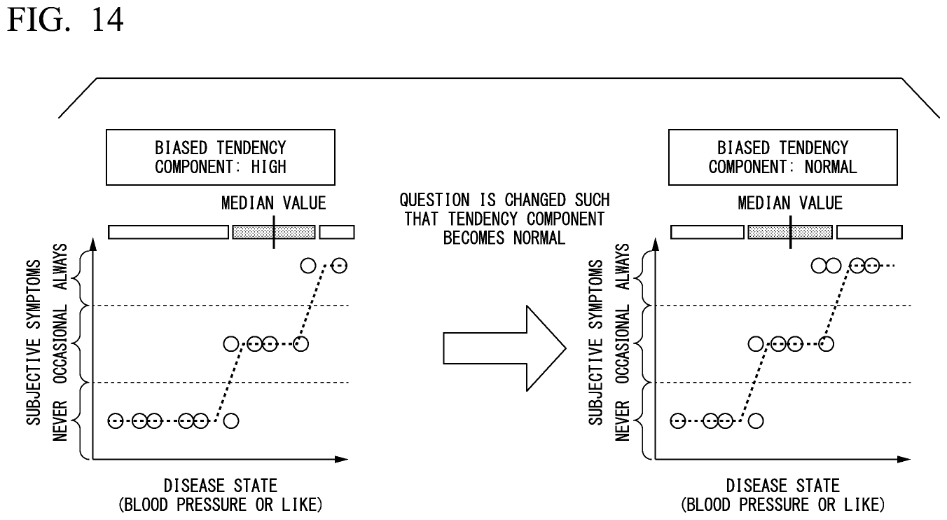

FIG. 13 is a diagram showing an example of an image IM50 including question content in a different questioning manner from that shown in FIG. 12. The image IM50 displays text information of a question different from that of the image IM40 for the same symptom item of “shortness of breath” as the image IM40. Specifically, the image IM50 displays question content such as “Do you ever feel that you have difficulty breathing?” Replies of the patient to the images IM40 and IM50 are acquired by the symptom reply acquisition function 141.

In this way, in the case of the same patient's disease state, it is possible to verify the reliability of a reply and improve the accuracy at the time of identifying a tendency component of the patient by changing the way of asking a question even for question content with respect to the same symptom. In addition, it is possible to correct differences in intervals of subjective symptoms for each patient on the basis of reply results for respective questions, thereby alleviating bias among patients. In addition, in a case where patient's replies have been obtained for a plurality of questions in different questioning manners for the same question content, the identification function 143 may identify component information using any reply only. For example, in a case where the same reply has been obtained for a plurality of questions in different questioning manners, the identification function 143 uses any reply only. Further, in a case where different replies have been obtained, the identification function 143 may adopt a reply that makes the component closer to “normal.”

In addition, the question generation function 147 may generate a question for adjusting component information for condition data to approach “normal” if the component information is not normal. FIG. 14 is a diagram showing generation of question content for adjusting component information. For example, if a median value is on a side on which a biased tendency component is high, as shown in FIG. 14, the question generation function 147 generates a question by changing question content or a questioning manner such that the tendency component becomes normal. In addition, the question generation function 147 may change representation of categories of subjective symptoms replied by a patient.

For example, the question generation function 147 adjusts representation of categories of subjective symptoms if a condition sensitivity component or a biased tendency component is high (or low). Further, the question generation function 147 elaborates description of a question such that replies are consistent, for example, if a random component is high. By adjusting question content in this way, it is possible to obtain replies with less bias for each patient, and in subjective symptoms influenced by various factors, the presence or absence of such influences and the factors can be more appropriately identified.

Further, in the above-described embodiment, instead of (or in addition to) subjective symptoms, objective symptoms that can be objectively attained by a person (e.g., a judge such as a doctor) other than a patient may be used. In this case, component information identified by the identification function 143 may include, as a fourth component, a tendency component specific to the judge in addition to the condition sensitivity component, the biased tendency component, and the random component.

In addition, a symptom of a patient in the above-described embodiments may include preferences and tastes regarding medical treatments. Further, regarding the condition sensitivity component and the biased tendency component among the above-described component information, data itself may be corrected or correction is performed at the time of displaying the data such that these components match between data. Further, the display control function 146 of the embodiment may visualize change in each component over time such that the change can be ascertained in a case where component information for each specific period is identified.

In the above-described embodiment, the symptom reply acquisition function 141 is an example of a “symptom reply acquisition unit,” the condition data acquisition function 142 is an example of a “condition data acquisition unit,” the identification function 143 is an example of an “identification unit,” the assignment function 144 is an example of an “assignment unit,” the image generation function 145 is an example of an “image generation unit,” the display control function 146 is an example of a “display control unit,” the question generation function 147 is an example of a “question generation unit,” and the notification function 148 is an example of a “notification unit.”

According to at least one embodiment described above, the medical information processing apparatus of the embodiment can more appropriately provide information on subjective symptoms of a patient influenced by various factors by including a symptom reply acquisition unit that acquires symptom reply information regarding a predetermined symptom of the patient, a condition data acquisition unit that acquires condition data of the patient related to the symptom, and an identification unit that identifies a condition sensitivity component of the symptom reply information on the basis of change in the symptom reply information with respect to change in the condition data.

Specifically, according to the embodiment, for example, in subjective symptoms influenced by various factors, by identifying the presence or absence of these influences and the factors, medical staff or a system can interpret subjective symptom questionnaire results in consideration of a bias even if there is the ease of awareness of patient's symptoms and a bias in a reply tendency between patients.

The above-described embodiment can be represented as follows.
A medical information processing apparatus including processing circuitry,
wherein the processing circuitry is configured to:
acquire symptom reply information regarding a predetermined symptom of a patient;
acquire condition data of the patient related to the symptom; and
identify a condition sensitivity component of the symptom reply information on the basis of change in the symptom reply information with respect to change in the condition data.

Although several embodiments have been described, these embodiments are presented as examples and are not intended to limit the scope of the invention. These embodiments can be implemented in various other forms, and various omissions, substitutions, and modifications can be made without departing from the spirit of the invention. These embodiments and modifications thereof are included in the scope and spirit of the invention, as well as the scope of the invention described in the claims and equivalents thereof.

What is claimed is:

1. A medical information processing apparatus comprising processing circuitry configured to:
acquire symptom reply information regarding a predetermined symptom of a patient;
acquire condition data of the patient related to the symptom; and
identify a condition sensitivity component of the symptom reply information on the basis of change in the symptom reply information with respect to change in the condition data.

2. The medical information processing apparatus according to claim 1, wherein the processing circuitry assigns the condition sensitivity component identified by an identification unit to the symptom reply information.

3. The medical information processing apparatus according to claim 1, wherein the processing circuitry displays the symptom reply information of the patient and the condition sensitivity component in association with each other.

4. The medical information processing apparatus according to claim 1, wherein the processing circuitry identifies a disease state interval in which the symptom reply information is not saturated, and identifies the condition sensitivity component on the basis of a ratio of the unsaturated interval to an entire interval in which symptom replies have been obtained.

5. The medical information processing apparatus according to claim 1, wherein the processing circuitry identifies a biased tendency component of replies of the patient for the symptom reply information.

6. The medical information processing apparatus according to claim 1, wherein the processing circuitry identifies a random component indicating inconsistency in replies of the patient for the symptom reply information.

7. The medical information processing apparatus according to claim 1, wherein the processing circuitry generates a question for obtaining symptom reply information from the patient and notifies the patient of the question in a case where the symptom reply information of the patient satisfies a specific condition.

8. The medical information processing apparatus according to claim 1, wherein the processing circuitry generates the same question content for the patient as question content in different questioning manners.

9. The medical information processing apparatus according to claim 1, wherein, in a case where component information for the condition data acquired by the condition data acquisition unit is not in a proper state, the processing circuitry generates a question for adjusting the component information to approach a proper state.

10. The medical information processing apparatus according to claim 9, wherein the processing circuitry adjusts representation of options for replies to the question in a case where the condition sensitivity component is not in a proper state.

11. The medical information processing apparatus according to claim 3, wherein the processing circuitry displays warning information according to the condition sensitivity component.

12. A medical information processing method, using a computer, comprising:
acquiring symptom reply information regarding a predetermined symptom of a patient;
acquiring condition data of the patient related to the symptom; and
identifying a condition sensitivity component of the symptom reply information on the basis of change in the symptom reply information with respect to change in the condition data.

13. A non-transitory computer-readable recording medium storing a program causing a computer to:
acquire symptom reply information regarding a predetermined symptom of a patient;
acquire condition data of the patient related to the symptom; and
identify a condition sensitivity component of the symptom reply information on the basis of change in the symptom reply information with respect to change in the condition data.
